(19) United States
(12) Patent Application Publication
(10) Pub. No.: US 2018/0260771 A1
(43) Pub. Date: Sep. 13, 2018

(54) Methods and Systems for Managing a Supply of Food Products

(71) Applicant: Walmart Apollo, LLC, Bentonville, AR (US)

(72) Inventors: Suman Pattnaik, Bentonville, AR (US); Steven Lewis, Bentonville, AR (US); Matthew Biermann, Fayetteville, AR (US)

(73) Assignee: Walmart Apollo, LLC, Bentonville, AR (US)

(21) Appl. No.: 15/919,007

(22) Filed: Mar. 12, 2018

Related U.S. Application Data
(60) Provisional application No. 62/470,013, filed on Mar. 10, 2017.

(51) Int. Cl. G06Q 10/08; G01N 33/02; G01N 27/02; G01N 21/84

(52) U.S. Cl. CPC G06Q 10/087; G01N 33/02; G01N 29/12; G01N 21/84; G01N 27/02

(57) ABSTRACT

Systems and methods are provided for managing an inventor of food products. The system includes a processor in communication with a memory storage device. The processor is configured to create a database of conditions of a food product available for purchase at a location, monitor one or more of the conditions of the food product at the location over a first period of time, determine the optimal condition of the food product based on the condition of the food product when sold at the location during the first period of time, and determine a state of the food products to be purchased for a second period of time later than the first period of time based on the determined optimal condition of the food product.

### FIELD OF THE INVENTION

The present invention relates to systems and methods for managing a supply of food products, and more particularly, for doing so in a retail setting.

### BACKGROUND OF THE INVENTION

Food products have various qualities that contribute to their overall condition. For example, an apple has a water content, a meat content, a seed content, an amount of seeds, a degree of ripeness, etc. Alternatively, a steak has a protein content, fat content, a degree of marbling, a degree of tenderness, etc. As such, although supplier may physically monitor their inventory of food products, they lack an efficient way to monitor their condition. As a result, in order to keep up with demand and cater to different expectations in food condition, suppliers in the food industry have to maintain a large inventory of food products. However, this results in significant waste.

Accordingly, systems and methods are needed to allow suppliers to more accurately monitor food products in their inventor and present optimal food products to purchasers. The present invention provides such systems and methods

### BRIEF SUMMARY OF THE INVENTION

In one aspect of the invention, a method for managing a supply of food products is provided. The method comprises creating a database of conditions of a food product available for purchase at a location, monitoring one or more of the conditions of a plurality of food products available for purchase at the location over a first period of time, determining an optimal condition of the food product based on the condition of the food product when sold at the location during the first period of time, and determining a state of the food products to be purchased for a second period of time later than the first period of time based on the determined optimal condition of the food product.

The monitoring of the condition of the food product and/or the determining of the optimal condition of the food product can comprise determining at least one of an external property and an internal property of the food product. Moreover, the determining of the external property of the food product can comprise receiving one or more images of an exterior region of the food product, and the determining of the internal property of the food product can comprise receiving one or more images of an interior region of the food product or a current differential between a pair of electrodes of a probe capable of being injected into the interior region of the food product. The internal property of the food product can comprise an amount of moisture in the food product.

The determining of the optimal condition of the food product can also comprise monitoring a number of food products sold at the location during the first period of time, determining a condition of each of said food products sold at said location during said first period of time, and calculating an average condition for said food products sold at said location during said first period of time based on the determined optimal condition of each of the food products when sold at said location during said first period of time. The average condition can be the optimal condition of the food products.

The state of the food product to be purchased can relate to one or more conditions of the food product, and the condition of the food production to be purchased can be less than the optimal condition.

In another aspect of the invention, a method for managing a supply of food products is provided, including creating a database of conditions of a food product available for purchase at a location, determining a time of optimal ripeness of the food product based on a condition of the food product when sold at the location, determining a number of the food products available for purchase at the location over a first period of time, estimating a number of the food products to be sold to at the location over the first period of time, and determining a state of the food products to be purchased for a second period of time later than the first period of time based on the determined time of optimal ripeness of the food product, the determined number of food products available for purchase at the location over the first period of time and the estimated number of sales of the food product at the location over the first period of time.

The method can also include monitoring one or more conditions of a plurality of food products available for purchase at the location over the first period of time. The monitoring of the condition of the food product and/or the determining of the time of optimal ripeness of the food product can comprise determining at least one of an external property and an internal property of the food product. Moreover, the determining of the external property of the food product can comprise capturing one more images of an exterior region of the food product. The determining of the internal property of the food product can comprise capturing one or more images of an interior region of the food product and determining a current differential between a pair of electrodes of a probe capable of being injected into the interior region of the food product. The internal property of the food product can comprise an amount of moisture in the food product.

The determining of the optimal condition of the food product can also comprise monitoring a number of food products sold at the location during the first period of time, determining a condition of each of said food products sold at said location during said first period of time, and calculating an average condition for said food products sold at said location during said first period of time based on the determined optimal condition of each of the food products when sold at said location during said first period of time. The average condition can be the optimal condition of the food products.

The state of the food product to be purchased can relate to one or more conditions of the food product, and the condition of the food production to be purchased can be less than the optimal ripeness of the food product.

In yet another aspect of the invention, a system for managing a supply of food product is provided, the system includes a memory storage device and a processor in communication with the memory storage device. The process can create a database of conditions of a food product available for purchase at a location, monitor one or more of the conditions of the food product available for purchase at the location over a first period of time, determine an optimal condition of the food product based on the condition of the food product when sold at the location during the first period of time, and determine a state and a time of the food products to be purchased for a second period of time later than the first period of time based on the determined optimal condition of the food product.

### BRIEF DESCRIPTION OF THE DRAWINGS

The features and advantages of the invention will be apparent from the following drawings wherein like reference numbers generally indicate identical, functionally similar, and/or structurally similar elements.

In the drawings:

FIG. 1 illustrates an exemplary system for managing food products in accordance with the embodiments of the present invention;

FIGS. 2 to 4 illustrate exemplary devices for analyzing an internal portion of a food product in accordance with the embodiments of the present invention;

FIGS. 5 and 6 illustrate exemplary optimal conditions for various food products in accordance with the embodiments of the present invention;

FIGS. 7 and 8 illustrate exemplary methods for managing food products at various location in accordance with the embodiments of the present invention;

FIG. 9 illustrates an exemplary system for managing food products at multiple locations in accordance with the embodiments of the present invention; and

FIG. 10 illustrates an exemplary server for managing food products in accordance with the embodiments of the present invention.

### DETAILED DESCRIPTION OF THE PRESENT INVENTION

Reference will now be made in detail to various embodiments of the present invention, examples of which are illustrated in the accompanying drawings. It is to be understood that the figures and descriptions of the present invention included herein illustrate and describe elements that are of particular relevance to the present invention. It is also important to note that any reference in the specification to “one embodiment,” “an embodiment” or “an alternative embodiment” means that a particular feature, structure or characteristic described in connection with the embodiment is included in at least one embodiment of the invention. As such, the recitation of “in one embodiment” and the like throughout the specification do not necessarily refer to the same embodiment.

The systems and methods disclosed herein are intended to be implemented for providing a supply of food products in an optimal condition. With the system analyzing one or more of internal and external properties of the food product, the systems and methods can determine the optimal condition of the food product, and can ensure such food products are available for purchase in the future. As such, the systems and methods can be implemented in a retail setting, but are not limited to use in a retail setting.

Condition as used herein refers to the internal and/or external properties of the food product.

Optimal condition as used herein refers to a condition of the food product that the purchaser prefers, or to an optimal ripeness of the food product.

Food product as used herein refers to any substance that can be used as food. For example, the food product can be meat, seafood, fish, fruit, vegetables or bread. More specifically, the food product can be a steak, salmon, strawberry, cantaloupe, watermelon, etc.

External property as used herein refers to any external characteristic related to the food product. For example, the external property can be a color, a shape, a size and a texture of the food product.

Internal property as used herein refers to any internal characteristic related to the food product. For example, the internal property can be protein content, fat content, seed content, water content, degree of ripeness, etc. More specifically, the internal property can be grams of protein, grams of fat, amount of seeds relative to the weight of food product, percentage of water content, degree of ripeness compared to average food product, estimated shelf life, etc.

Along these lines, the internal and/or external properties of each food product may not be identical, and they may be dependent on the identity of the food product. As such, each food product may have one or more internal and/or external properties unique to its identity. For example, a strawberry may have one or more internal and external properties unique to itself, and those properties maybe different than those of poultry. Along these lines, the internal and/or external properties of each food product may be based on whether the food is organic or non-organic. For example, where the food product is organic, the internal property can be the amount of pesticides, fertilizers, chemical preservatives and monosodium glutamate (MSG) in the food product.

Retail setting s used herein refers to any online or brick-and-mortar outlet selling one or more food products to the public including, but not limited to, grocery stores, department stores, supermarkets, hypermarkets, warehouse stores, specialty stores, retail store, etc.

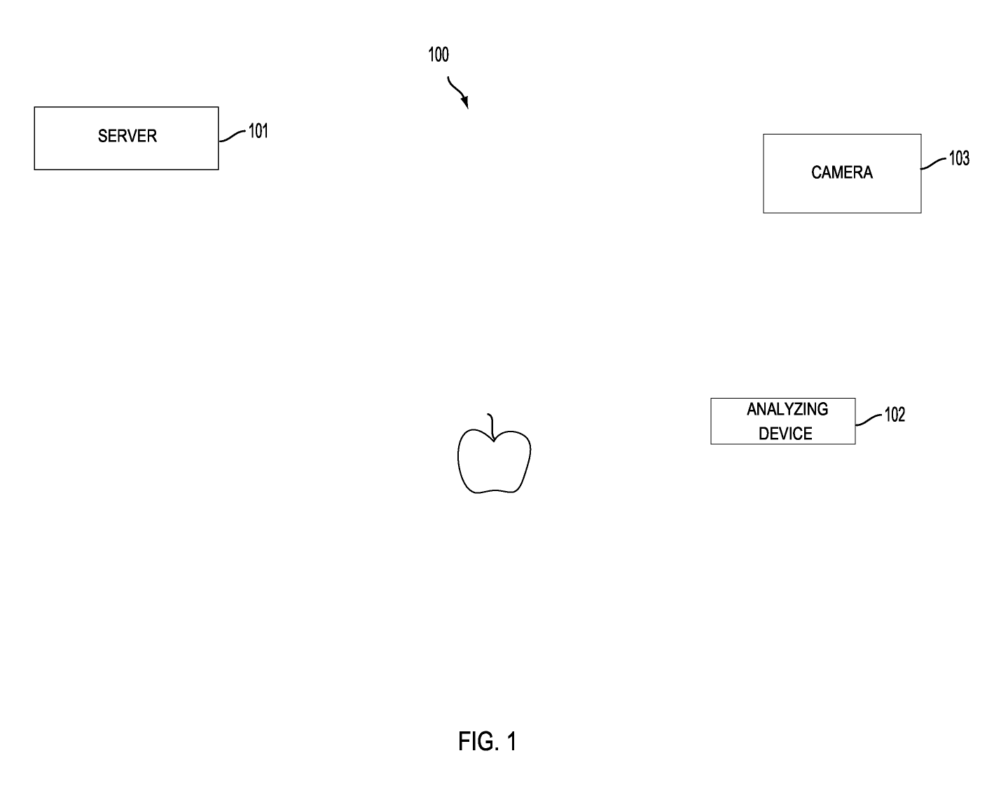

Referring now to the figures, various exemplary embodiments of systems and methods for providing optimal food products to customers in a retail setting and managing an inventory of food products in the retail setting will be described. Referring now to FIG. 1, a system 100 for implementation in a retail setting is provided. As shown, the system 100 can include a server 101, one or more analyzing devices 102 and one or more cameras 103.

The Camera

As illustrated in FIG. 1, the camera 103 can be wirelessly connected to the server 101. The camera 103 can take one or more pictures of an exterior region of one or more food products, and can do so automatically and/or at specified times. For example, the camera 103 can automatically take one or more pictures of food products at 7:00 AM every day and/or at times of utilization of the analyzing device 102. Along these lines, the camera 103 can also take one or more pictures of the exterior region of the food product upon indication by an individual.

The Analyzing Device

The analyzing device 102 can be in communication with the server 101 and/or the camera 103. The analyzing device 102 can monitor and/or analyze the interior region of the food product in any number of ways.

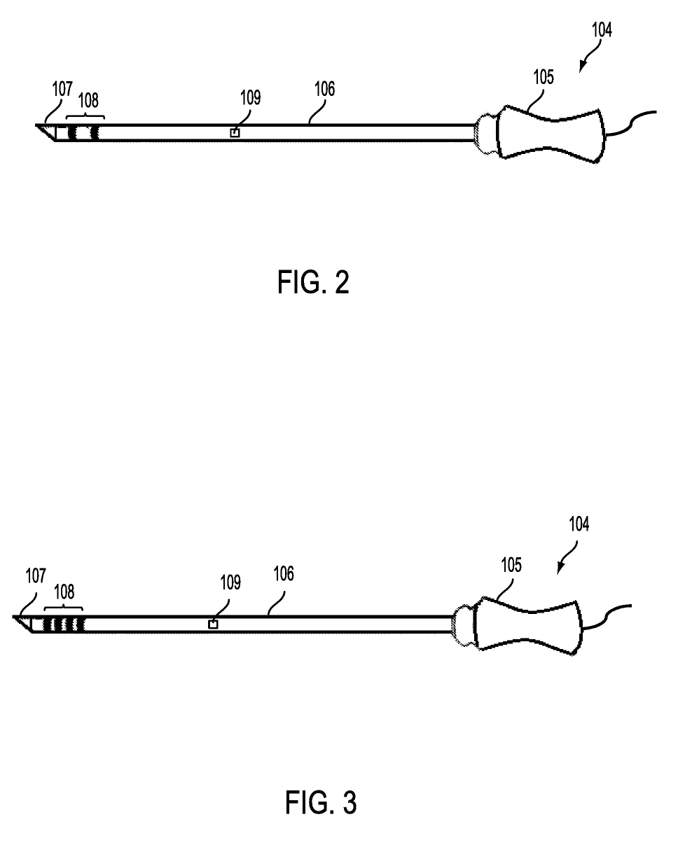

In some embodiments, as illustrated in FIGS. 2 and 3, the analyzing device 102 (illustrated in FIG. 1) can be a probe 104. The probe 104 can comprise a handle 105 for the user to grab, an elongated structure 106 attached to and extending outwardly from a first end of the handle 105, and a sharp tip 107 attached to and outwardly extending an end of the elongated structure opposite from the handle 105. The elongated structure 106 can further comprise a sensor 109 configured to send a signal to the user to indicate that the user has inserted a sufficient portion of the elongated structure into the food product. Moreover, the elongated structure 106 can be of a size and strength to allow it to be easily inserted into the food product without significant difficulty by the user, and to not cause a hole in the food product noticeable by the naked eye.

Furthermore, the elongated structure 106 of the probe 104 may comprise one or more electrodes 108 configured to determine an impedance of an interior region of the food product. According to an embodiment, as illustrated in FIG. 2, the probe 104 can comprise an elongated structure 106 having two electrodes 108 spaced from each other at an end of the probe 104 to be inserted into the food product. According to another embodiment, as illustrated in FIG. 3, the elongated structure 106 may have multiple pairs of electrodes 108 utilized to measure an impedance between various interior regions of the food product. In each of these embodiments, the central unit 103 (illustrated in FIG. 1) can be configured to transmit an electrical signal to the electrode(s) of each elongated structure, and to measure an impedance of an interior region between each pair of electrodes 108.

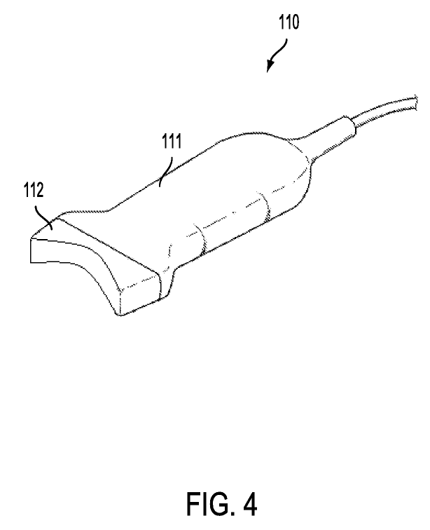

According to yet another embodiment, as shown in FIG. 4, the analyzing device 102 (illustrated in FIG. 1) can be an ultrasound device 110. The ultrasound device 110 can have a first end 111 and a second end 112. The first end 111 can comprise a handle 105 for the user to grab. The second end 112 can be configured to direct frequencies to the food product. As such, as illustrated, the second end 112 can be arc shaped.

Upon receipt of an electrical signal from the central device, the ultrasound device 110 sends sound waves having low frequencies for a predetermined amount of time to the food product. Preferably, the frequency is in the range of 100 kHz and 1 W/cm². The sounds echoes are recorded by the central device and capable of being transformed to an image of the inside of the food product. As such, the ultrasound device 110 is not intrusive and may be considered preferable to the probe 104 (illustrated in FIGS. 2 and 3).

According to yet a further embodiment, the probe 104 and ultrasound device 110 can both be used to determine the internal properties of the food product. They can be used in conjunction with each other to provide a more accurate representation of the internal properties of the food product.

The Server

The server 101 can be in communication with the camera 103 and/or analyzing device 102. As such, the server 101 can be configured to create and maintain a database of food products and one or more internal and/or external properties relating to each food product.

The server 101 can monitor one or more conditions of the food products, particularly over a first period of time. The first period of time can be predefined by a user or the server 101. As such, the first period of time can be any definite period of time including, but limited, to a one week period, a two-week period, a month period, etc.

To determine the condition of the food products, the server 101 can utilize information from the one or more pictures taken by the camera 103 to determine one or more of an identity of a food product, an amount of food products in a predefined area, and an external property of the food product. The external property, such as a color or deformations, of the food product can be indicative of the condition of the food product (i.e., ripeness) and its identity.

The server 101 can also utilize information received from the analyzing device 102 to determine one or more internal properties of the food product, particularly over the first period of time. For example, according to an embodiment, where the analyzing device 102 comprises a probe having an elongated structure bearing two electrodes, the server 101 can determine one or more internal properties based on an impedance across an interior region of the food product between the electrodes. According to another embodiment, where the analyzing device 102 comprises an ultrasound device, the server 101 can determine one or more internal properties based on an image of an interior portion of the food product. According to yet another embodiment, where the analyzing device 102 comprises a probe and an ultrasound device, the server 101 can determine one or more internal properties based on the impedance across the electrodes and the image of the interior portion of the food product.

The server 101 can receive the information from the analyzing device 102 at predetermined times. According to an embodiment, the analyzing device 102 can send the results to the server 101 only when it is used. According to another embodiment, the analyzing device 102 can send the results to the server 101 only when the food product is purchased.

As such, the server 101 can also automatically determine an identity and one more internal properties of the food product through utilization of the analyzing device 102 and/or camera 103. According to an embodiment, the server 101 can determine the food type (i.e., watermelon) based on the information received from the camera 103 and, thereafter, determine one or more internal properties related to the food product (i.e., seed content, percentage of water, percentage of meat, degree of ripeness, age, shelf life, etc.) based on information received from the analyzing device 102. According to another embodiment, the server 101 can determine the food type and one or more internal properties relating thereto based solely on information received from the analyzing device 102.

The server 101 can further determine one or more optimal conditions of the food product based on the condition of the food product when it is purchased, particularly over the first period of time. As stated previously, the optimal condition can refer to a condition of the food product that the purchaser prefers. As such, the optimal condition can be reflective of the customer's preferred internal and/or external properties of the food product. Alternatively, as also stated previously, the optimal condition can be reflective of an optimal ripeness of the food product.

To determine the optimal conditions of the food product, the server 101 can determine a condition of a number of food products sold at a location over the first period of time. Thereafter, the server 101 can determine one or more groups for each food product based on the determined optimal conditions. The groups can be indicative of multiple different optimal conditions of the food product. Subsequently, the server 101 can calculate an average condition for the food products of each group.

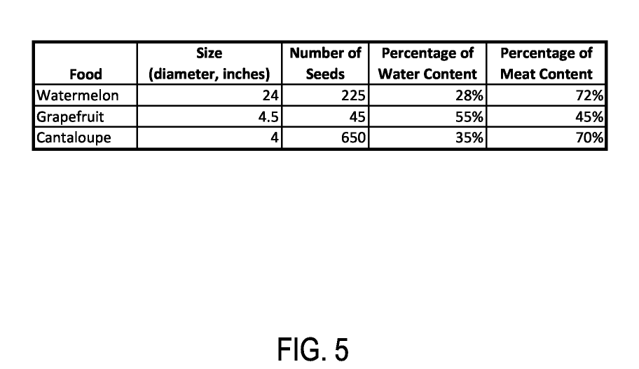

According to an exemplary embodiment, as shown in FIG. 5, the server 101 can determine a single optimal condition for a watermelon, a grapefruit, and a cantaloupe over a first period of time. Specifically, the server 101 can determine the optimal condition for a watermelon is one that is approximately 24 inches in diameter and has approximately 225 seeds, 30% water content, and 70% meat content.

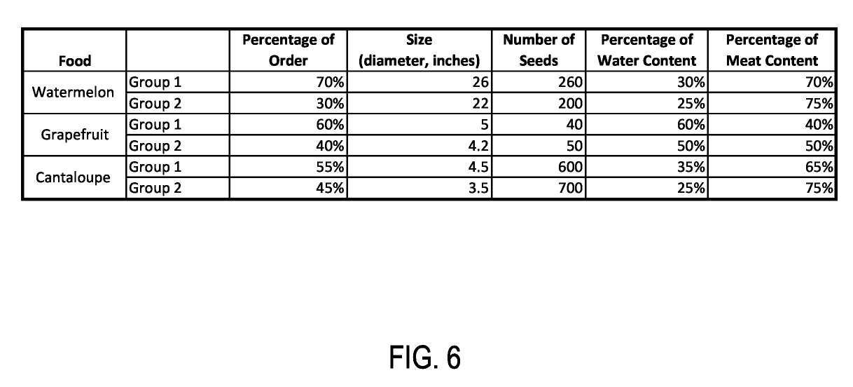

Accordingly to another exemplary embodiment, as shown in FIG. 6, the server 101 can determine multiple different optimal conditions for a watermelon, a grapefruit, and a cantaloupe. Specifically, the server 101 can determine that a first optimal condition for a watermelon is one that is approximately 26 inches in diameter and has approximately 260 seeds, 30% water content, and 70% meat content, and the second optimal condition for a watermelon is one that is approximately 22 inches in diameter and has approximately 200 seeds, 25% water content, and 75% meat content.

The server 101 can further determine a weighted average to each optimal condition for each food product. The weighted average can refer to the importance of each optimal condition. As such, the weighted average can be reflective of each optimal condition relative to one another, and can be assigned a percentage relative to the total. For example, as illustrated in FIG. 6, the server 101 can determine that there are the first and second optimal conditions for the watermelon are 70% and 30%, respectively.

Moreover, the server 101 can determine a number and/or state of food products available for purchase, particularly over the first period of time. The number and/or state of the food products currently available for purchase can be determined from information received from the camera 103 and/or analyzing device 102. According to an embodiment, the server 101 can determine a number of food product currently available for purchase based on previous orders and/or images taken by the camera 103, and can determine a state of the food product based on one or more images taken by the camera 103 and/or information received from analyzing device 102.

Along these lines, the server 101 can estimate a number of the food products that are likely to be sold, particularly over the first period of time. The estimate of the number of the food products to be sold can be based on prior sales including, but not limited to, one or more of sales of previous periods of time equal to the first period of time. Along these lines, the estimate of the number of the food products to be sold can be based on the season. For example, particular food products may be known to be “in-season,” thereby increasing their demand.

Based on the optimal condition(s), the server 101 can determine a state of food products to be purchased, particularly for a second period of time later than the first period of time. The second period of time may be equal or less than the first period of time. Moreover, the state of the food product relates to one or more conditions of the food product and can be less than the optimal condition. As such, it may be advantageous to purchase food products that are less than the optimal condition based on the food product's self-life and their estimated time of arrival. By requesting food products in less than their optimal condition, they can be received and made available in their optimal condition. Along these lines, the server 101 can determine different states of food products to be purchased, particularly for a second period of time later than the first period of time, based on the different determined optimal conditions.

Moreover, the server 101 can determine a number of food products to be purchased in the determined state, particularly for the second period of time. The number of food products to be purchased in the determined state can be based on a number and a state of food products currently in stock. As stated previously, the number and state of the food products currently in stock can be determined from information received from the camera 103 and analyzing device 102. As also stated previously, according to an embodiment, the server 101 can determine a number of food product currently in stock based on previous orders and/or images taken by the camera 103, and can determine a state of the food product based on one or more images taken by the camera 103 and/or information received from analyzing device 102.

Subsequently, the server 101 can provide a report of a state and/or a number of said food products to be purchased, particularly for the second period of time later. Along these lines, the server 101 can send a request to a food provider for the number and/or state of the food products. The server 101 can continually do so over multiple periods of times. Alternatively, the server 101 can do so upon indication by an individual.

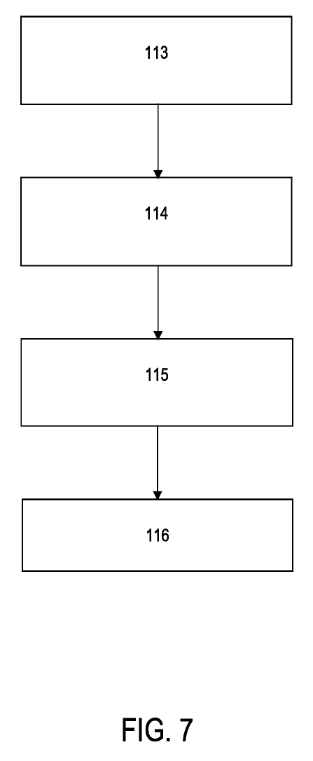

Referring now to FIG. 7, an exemplary method for managing a supply of food products is presented that can be performed in accordance with one or more embodiments of the present invention. First, at block 113, a database of conditions of a food product available for purchase at a location over a first period of time is created. Next, at block 114, one or more of the conditions of the food product at the location are monitored over the first period of time. Subsequently, at block 115, an optimal condition of the food product is determined based on the condition of the food product when it is sole at the location during the first period of time. Thereafter, at block 116, a state of the food products to be purchased for a second period of time later than the first period of time is determined based on the determining of the optimal condition of the food product. Each of these steps can be processed in accordance with one or more embodiments of the invention as described above.

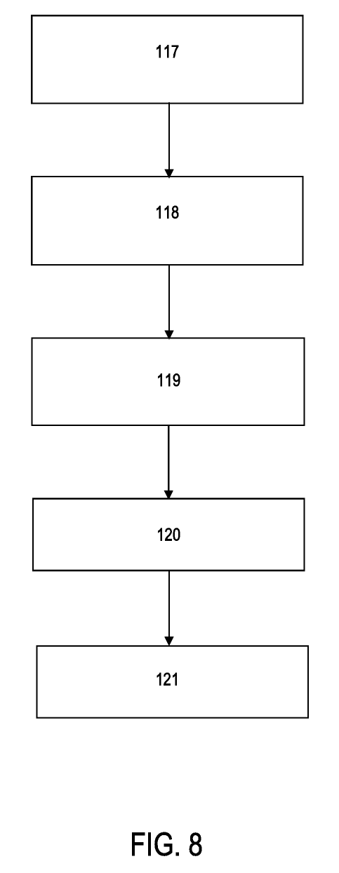

Referring now to FIG. 8, another exemplary method for managing a supply of food products is presented that can be performed in accordance with one or more embodiments of the present invention as discussed above. First, at bock 117, a database of conditions of a food product is created. Thereafter, at block 118, a time of optimal ripeness of the food product is determined based on a condition of the food product when sold at the location. Next, at block, 119, a number of the food products available for purchase at the location over a first period of time is determined. Subsequently, at block 120, a number of the food products to be sold to over the first period of time is estimated. Afterward, at block 122, a state of the food products to be purchased for a second period of time later than the first period of time is determined based on the determined time of optimal ripeness of the food product, the determined number of food products available for purchase at the location over the first period of time and the estimated amount of sales of the food product at the location over the first period of time. Each of these steps can be processed in accordance with one or more embodiments of the invention as described above.

Regression analysis may be used to determine the properties of the item when it is purchased by a customer. These properties may be correlated with a number of the same items sold over time in order to identify those properties of the item that are present when customers purchase the item. For example, customers may often purchase a watermelon when in makes a solid sound when “thumped” with a finger. The analysis tracks and records this type of data gathered as described above. Regression analysis performed using the various parameters and physical properties of the item for a number of customer transactions provides a model expression to predict the quality of food products and recommend the optimal time of receipt and quality.

In an example, the regression analysis may have one or more of the following inputs:
1) Duration, a particular food product (e.g., a fruit like water melon or cantaloupe or apple etc.) have spent in the store from the time of receipt.
2) Purchase time from the time of receipt
3) Overall color at the point of receipt
4) Overall frequency of response sound to ultra violet emission (Mimicking the finger “thump” process many consumers do on watermelon)
5) Overall Weight
6) Processing type (Natural vs manmade, e.g. in a case of similar to bananas)
7) Optional Packaging, Route to store and placement in store parameters
   Additional properties of the item may also be included, or substituted in place of the above.

Once identified, the qualities customers like to see in the item may be used to determine when items currently in the store may be sold. The inventory system may predict the sales rate for the item, and determine when additional items should be ordered in order to maintain ideal inventory levels. For example, a batch of items, watermelons, may be received at a store. A sample number of the items may be analyzed. If the sample indicates that the watermelons are at or near the optimum selling point, the inventory system can automatically order new inventory. If the sample indicates that the items have yet to reach the selling point, the inventory system is freed up to process other tasks, improving speed and efficiency of the system. Items or a subset of the items may be analyzed when received and at other points, for example, after being on a shelf for predetermined period of time. Inventory and order decisions may be automated based on the regression analysis and model.

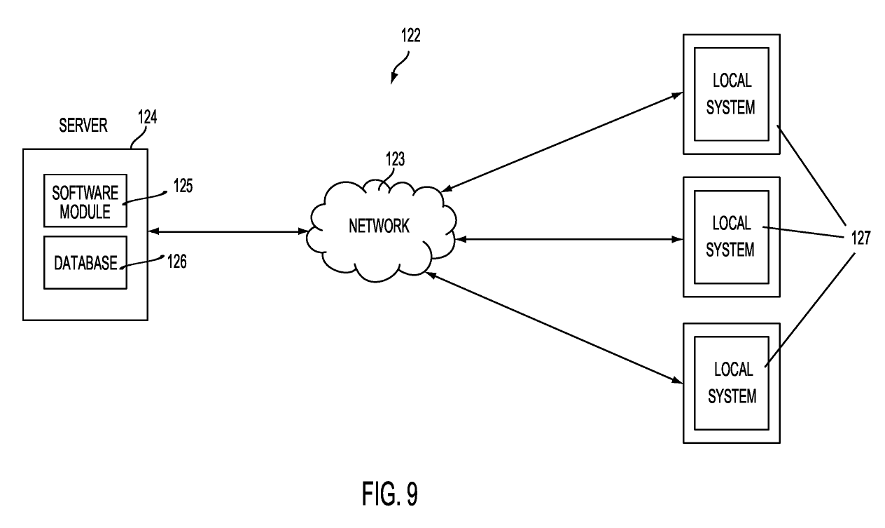

Referring now to FIG. 9, a diagram of an exemplary system 122 is shown that may be utilized in accordance with one or more embodiments of the present invention as discussed above. System 122 can include a network 123, server 124, software module 125, database 126 and one or more local systems 127. The local system 127 can comprise a server, an analyzing device, and a camera as described above. The local system 126 and the server 124 can be coupled to a network 123 and configured to send and/or receive data to the network 123. According to an embodiment, the components of each of the local systems 127 can communicate with the server 124 over the network 123 to determine the optimal condition of a food product.

Network 123 can provide network access, data transport and other services to the devices coupled to it in order to send/receive data from any number of user devices, as explained above. In general, network 123 can include and implement any commonly defined network architectures including those defined by standard bodies, such as the Global System for Mobile Communication (GSM) Association, the Internet Engineering Task Force (IETF), and the Worldwide Interoperability for Microwave Access (WiMAX) forum.

Server 124 can also be any type of communication device coupled to network 123, including but not limited to, a personal computer, a server computer, a series of server computers, a mini computer, and a mainframe computer, or combinations thereof. Server 124 can be a web server (or a series of servers) running a network operating system. Server 124 can be used for and/or provide cloud and/or network central.

Database 126 can be any type of database, including a database managed by a database management system (DBMS). A DBMS is typically implemented as an engine that controls organization, storage, management, and retrieval of data in a database. DBMSs frequently provide the ability to query, backup and replicate, enforce rules, provide security, do computation, perform change and access logging, and automate optimization.

Software module 125 can be a module that is configured to send, process, and receive information at server 124. Software module 125 can provide another mechanism for sending and receiving data at server 124 besides handling requests through web server functionalities.

Although software module 125 can be described in relation to server 124, software module 125 can reside on any other device. Further, the functionality of software module 125 can be duplicated on, distributed across, and/or performed by one or more other devices, either in whole or in part.

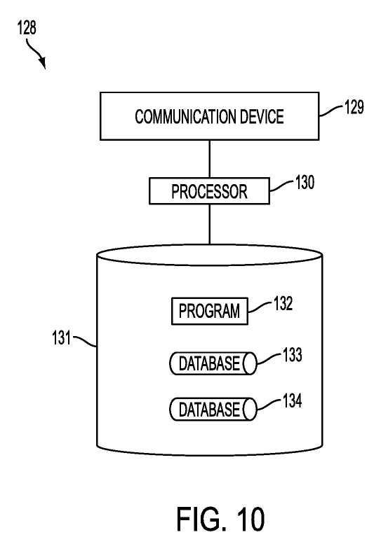

Referring now to FIG. 10, a schematic diagram of an exemplary server 128 is illustrated that may utilized in accordance with one or more embodiments of the present invention as discussed above. The exemplary server 128 includes a processor 130, a communication device 129 and a data storage or memory component 131. The processor 130 is in communication with both the communication device 129 and the memory component 131. The communication device 129 may be configured to communicate information via a communication channel, wired or wireless, to electronically transmit and receive digital data related to the functions discussed herein. The communication device 129 may also be used to communicate, for example, with one or more human readable display devices. The memory component 131 may comprise any appropriate information memory component, including combinations of magnetic memory components (e.g., magnetic tape, radio frequency tags, and hard disk drives), optical memory components, computer readable media, and/or semiconductor memory devices. The memory component 131 may store the program 131 for controlling the processor 130. The processor 130 performs instructions of the program 131, and thereby operates in accordance with the present invention. The memory component 131 may also store and send all or some of the information sent to the processor 130 in one or more databases 133 and 134.

Communication device 129 may include an input device including any mechanism or combination of mechanisms that permit an operator to input information to communication device 129. Communication device 129 may also include an output device that can include any mechanism or combination of mechanisms that outputs information to the operator.

While various exemplary embodiments have been described above, it should be understood that they have been presented by way of example only, and not limitation. Thus, the breadth and scope of the present disclosure should not be limited by any of the above-described exemplary embodiments.

Although the foregoing description is directed to the preferred embodiments of the invention, it is noted that other variations and modifications will be apparent to those skilled in the art, and can be made without departing from the spirit or scope of the invention. Moreover, features described in connection with one embodiment of the invention can be used in conjunction with other embodiments, even if not explicitly stated above.

We claim:

1. A method for managing a supply of food products, the method comprising:
creating, by a computer, a database of conditions of a food product available for purchase at a location;
monitoring, by said computer, one or more of said conditions of said food product at said location over a first period of time;
determining, by said computer, an optimal condition of said food product based on said condition of said food product when sold at said location during said first period of time; and
determining, by said computer, a state of said food products to be purchased for a second period of time later than said first period of time based on the determined optimal condition of said food product.

2. The method of claim 1, wherein at least one of the monitoring of said condition of said food product and the determining of the optimal condition of the food product comprises determining at least one of an external property and an internal property of said food product.

3. The method of claim 2,
wherein the determining of the external property of said food product comprises receiving one or more images of an exterior region of said food product, and
wherein the determining of the internal property of said food product comprises receiving one or more images of an interior region of said food product or a current differential between a pair of electrodes of a probe capable of being injected into said interior region of said food product.

4. The method of claim 3, wherein the internal property of said food product comprises an amount of moisture in said food product.

5. The method of claim 1, wherein the determining of the optimal condition of the food product comprises:
monitoring, by said computer, a number of food products sold at said location during said first period of time; and
determining, by said computer, a condition of each of said food products sold at said location during said first period of time; and
calculating, by said computer, an average condition for said food products sold at said location during said first period of time based on the determined optimal condition of each of said food products when sold at said location during said first period of time,
wherein the average condition is the optimal condition of said food products.

6. The method of claim 1, wherein the state of said food product to be purchased relates to one or more conditions of said food product.

7. The method of claim 6, wherein the condition of the food production to be purchased is less than the optimal condition.

8. The method of claim 1, additionally comprising:
determining a number of said food products to be purchased for said second period of time based on a number and a state of said food products currently in stock.

9. The method of claim 8, wherein the determining of the number of food products to be purchased for the second period of time is further based on historical data, the historical data including a number of said food products sold over a similar period of time to said second period of time in one or more previous years.

10. The method of claim 1, additionally comprising:
sending, by said computer, a request to a food product provider for said number and state of said food products for said second period of time.

11. A method for managing a supply of food products, the method comprising:
creating, by a computer, a database of conditions of a food product available for purchase at a location;
determining, by said computer, a time of optimal ripeness of said food product based on a condition of said food product when sold at said location;
determining, by said computer, a number of said food products available for purchase at said location over a first period of time;
estimating, by said computer, a number of said food products to be sold at said location over said first period of time; and
determining, by said computer, a state and a number of said food products to be purchased for a second period of time later than said first period of time based on the determined time of optimal ripeness of said food product, the determined number of food products available for purchase at said location over said first period of time and the estimated number of sales of said food product at said location over said first period of time.

12. The method of claim 11, additionally comprising:
monitoring, by said computer, one or more conditions of a plurality of food products available for purchase at said location over said first period of time,
wherein at least one of the monitoring of said condition of said food product and the determining of the time of optimal ripeness of the food product comprises determining at least one of an external property and an internal property of said food product.

13. The method of claim 12,
wherein the determining of the external property of said food product comprises capturing one more images of an exterior region of said food product, and
wherein the determining of the internal property of said food product comprises at least one of:
capturing one or more images of an interior region of said food product, and
determining a current differential between a pair of electrodes of a probe capable of being injected into said interior region of said food product.

14. The method of claim 13, wherein the internal property of said food product comprises an amount of moisture in said food product.

15. The method of claim 11, wherein the determining of the time of optimal ripeness of the food product comprises:
monitoring, by said computer, a number of food products sold at said location during said first period of time;
determining, by said computer, a condition of each of said food products sold at said location during said first period of time; and
calculating, by said computer, an average condition for said food products sold at said location during said first period of time based on the determined condition of each of said food products when sold at said location during said first period of time,
wherein the average condition is the optimal ripeness of said food products.

16. The method of claim 11, wherein the state of said food product to be purchased relates to one or more conditions of said food product.

17. The method of claim 16, wherein the condition of the food production to be purchased is less than optimal ripeness.

18. The method of claim 11, additionally comprising:
determining a number of said food products to be purchased for said second period of time based on a number and a state of said food products currently in stock.

19. The method of claim 18, wherein the determining of the number of food products to be purchased for the second period of time comprises performing a regression analysis on properties of the food product for a number of customer transactions to determine a model expression related to quality of the food product, wherein the properties include:
time of receipt of the food product;
purchase time from the time of receipt;
overall color at the point of receipt;
overall frequency of response sound to ultra violet emission;
overall weight;
processing type; and
optional packaging, route to store and placement in store parameters.

20. A system for managing a supply of food products, the system comprising:
a memory storage device; and
a processor in communication with said memory storage device and configured to:
create a database of conditions of a food product available for purchase at a location;
monitor one or more of said conditions of said food product at said location over a first period of time;
determine an optimal condition of said food product based on said condition of said food product when sold at said location during said first period of time; and
determine a state of said food products to be available for purchase over for a second period of time later than said first period of time based on the determined optimal condition of said food product.
